(19) United States
(12) Patent Application Publication
(10) Pub. No.: US 2025/0270504 A1
(43) Pub. Date: Aug. 28, 2025

(54) METHODS FOR MANUFACTURING AND USING IMMUNE-TOLERIZED FETAL STEM CELL-DERIVED EXTRACELLULAR VESICLES FOR OSTEOARTHRITIS TREATMENT

(71) Applicant: STEMMEDICARE CO., LTD., Seoul (KR)

(72) Inventor: Jang Ho LEE, Seoul (KR)

(73) Assignee: STEMMEDICARE CO., LTD., Seoul (KR)

(21) Appl. No.: 19/059,721

(22) Filed: Feb. 21, 2025

(30) Foreign Application Priority Data
Feb. 22, 2024 (KR) 10-2024-0025944

(51) Int. Cl. C12N 5/0735; A61K 35/545; A61P 19/02

(52) U.S. Cl. CPC C12N 5/0606; A61K 35/545; A61P 19/02; C12N 2502/025; C12N 2502/1317; C12N 2502/1358; C12N 2523/00; C12N 2527/00

(57) ABSTRACT

Disclosed are immune-tolerized fetal stem cell-derived extracellular vesicles. These are immune-tolerized fetal stem cell-derived extracellular vesicles contain fibroblast growth factor-2 (FGF-2), fibroblast growth factor-7 (FGF-7), insulin-like growth factor-1 (IGF-1), transforming growth factor-beta 1 (TGF-β1), bone morphogenetic protein-4 (BMP-4), bone morphogenetic protein-5 (BMP-5), and bone morphogenetic protein-7 (BMP-7), and include HLA-G protein.

### TECHNICAL FIELD

The present invention relates to the use of immune-tolerized fetal stem cell-derived extracellular vesicles as a pharmaceutical composition for osteoarthritis treatment, a method for manufacturing the pharmaceutical composition, and a composition for osteoarthritis treatment using the pharmaceutical composition. In particular, the present invention relates to the use of immune-tolerized fetal stem cell-derived extracellular vesicles as a pharmaceutical composition for osteoarthritis treatment, which have hyaline cartilage regeneration and anti-inflammatory efficacies and prevent immune rejection due to allogeneic transplantation, a method for manufacturing the pharmaceutical composition, and a composition for osteoarthritis treatment using the pharmaceutical composition.

### BACKGROUND ART

The information contained in this background art section has been prepared to enhance the understanding of the background of the invention and may include matters that are not prior art already known to those skilled in the art to which this technology belongs.

Osteoarthritis (OA), commonly known as degenerative arthritis, is a representative degenerative disease in which the cartilage tissue that makes up the joint is gradually and irreversibly destroyed, resulting in structural changes in the bone, inflammatory response, and joint pain.

Due to the rapid aging trend worldwide, osteoarthritis is the most common degenerative disease, affecting more than 35% of the population over 60 years of age, and aging is the most representative cause of osteoarthritis. Osteoarthritis occurs due to irreversible cartilage degeneration caused by failure of homeostatic control of cartilage tissue to restore cartilage tissue damage caused by genetic factors, obesity, and physical factors such as trauma in addition to this.

There are yet no approved disease-modifying osteoarthritis drugs (DMOADs) that not only relieve joint pain and improve function, but also inhibit or reverse structural disease progression. Analgesics and hyaluronic acid injections, which only have temporary pain relief efficacy, are used in the treatment of more than 60% of osteoarthritis cases, but have limitations such as causing various side effects and decreased efficacy due to drug resistance. In addition, artificial joint replacement surgery is mainly used for patients with moderate to severe osteoarthritis, but the social demand for the development of new osteoarthritis drugs is gradually increasing because of the disadvantages such as risk of infection, thrombosis, reoperation due to limited lifespan, high cost, long-term patient discomfort, and serious functional impairment in elderly people.

Recently, cell therapy products using autologous and allogeneic chondrocytes or stem cells have been commercialized as osteoarthritis drugs with anti-inflammatory and cartilage regeneration efficacies. However, in the case of autologous chondrocytes, there is a limitation that autologous chondrocytes can only be applied to patients under 55 years of age. Contrary to expectations, allogeneic stem cell therapy products using mesenchymal stem cells derived from adipose tissue, bone marrow, and umbilical cord blood have only been recognized for their inflammation relief efficacy. Meanwhile, as side effects such as immune rejection by allogeneic cells, risk of cancer formation, promotion of tissue calcification, and decreased therapeutic efficacy due to low survival rate have been revealed, extracellular vesicles that can replace cell therapy products are attracting attention.

Extracellular vesicles, commonly known as exosomes, are nano-sized vesicles with a double lipid membrane structure that are secreted outside of living cells, and play a role in delivering various physiologically active substances, such as proteins, enzymes, and nucleic acids to other cells or tissues.

According to recently reported study results, stem cell-derived extracellular vesicles are known to have the efficacies of reducing inflammatory responses, inhibiting and preventing cartilage degradation, restoring cartilage extracellular matrix and maintaining homeostasis, inducing chondrocyte differentiation, and regenerating cartilage tissue. However, most of the chondrocytes regenerated by extracellular vesicles derived from adult stem cells or cartilage tissue are degenerative fibrous cartilage that has weaker physical strength than hyaline cartilage, which is normal cartilage tissue. It has recently been reported that extracellular vesicles secreted from non-autologous cells can induce undesirable immune rejection from the patient's immune system due to mismatch in the major histocompatibility complex (MHC) antigens of the originating cell present in the double lipid membrane, just like allogeneic cell therapy products.

### CITATION LIST

### Patent Documents

Patent Document 1: U.S. Pat. No. 11,566,220 B2

Patent Document 2: U.S. Pat. No. 11,771,720 B2

Patent Document 3: U.S. Pat. No. 12,150,962 B2

### DISCLOSURE

### Technical Problem

An object of the present invention is to provide a pharmaceutical composition for osteoarthritis treatment, containing immune-tolerized fetal stem cell-derived extracellular vesicles containing various components that promote hyaline cartilage regeneration and relieve inflammation at higher levels than stem cell-derived extracellular vesicles obtained under general culture conditions, and thus exhibit superior hyaline cartilage regeneration and anti-inflammatory efficacies; a method for manufacturing the extracellular vesicles; and a composition for osteoarthritis treatment using the extracellular vesicles.

Another object of the present invention is to provide a pharmaceutical composition for osteoarthritis treatment, containing immune-tolerized fetal stem cell-derived extracellular vesicles, which can deliver active ingredients completely into target cells and tissues without causing immune rejection by autologous extracellular vesicles and can be thus used as fundamental disease-modifying osteoarthritis drugs (DMOADs) without side effects; a method for manufacturing the pharmaceutical composition; and a composition for osteoarthritis treatment using the pharmaceutical composition.

### Technical Solution

The pharmaceutical composition for osteoarthritis treatment, containing immune-tolerized fetal stem cell-derived extracellular vesicles according to an embodiment of the present invention contains fibroblast growth factor-2 (FGF-2), fibroblast growth factor-7 (FGF-7), insulin-like growth factor-1 (IGF-1), transforming growth factor-beta 1 (TGF-β1), bone morphogenetic protein-4 (BMP-4), bone morphogenetic protein-5 (BMP-5), and bone morphogenetic protein-7 (BMP-7), and includes HLA-G protein.

Here, the extracellular vesicles additionally contain interleukin-1 receptor antagonist (IL-1ra), interleukin-4 (IL-4), interleukin-10 (IL-10), and interleukin-13 (IL-13).

The extracellular vesicles additionally contain tissue metalloproteinase inhibitor 1 (TIMP1) and tissue metalloproteinase inhibitor 2 (TIMP2).

The extracellular vesicles additionally contain miR-26a, miR-26b, miR-92a, miR-127, miR-136, miR-140, miR-146a, and miR-148.

The extracellular vesicles additionally contain aggrecan, collagen II, proteoglycan 4 (PRG4), and sex-determining region Y (SRY)-related protein 9 (SOX9).

The extracellular vesicles additionally contain human leukocyte antigen-G1 (HLA-G1), human leukocyte antigen-G2 (HLA-G2), human leukocyte antigen-G5 (HLA-G5), and human leukocyte antigen-G6 (HLA-G6).

The fetal stem cells are amniotic fluid-derived fetal stem cells.

The method for manufacturing immune-tolerized fetal stem cell-derived extracellular vesicles for osteoarthritis treatment according to an embodiment of the present invention includes: a first step of obtaining extracellular vesicles containing cartilage extracellular matrix through the co-culture of human chondrocytes and bone marrow-derived mesenchymal stem cells; a second step of obtaining extracellular vesicles derived from immune-tolerized trophoblasts that continuously express and secrete HLA-G; and a third step of inoculating fetal stem cells into an ex vivo culture matrix gel and subculturing the cells in a serum-free medium containing the human chondrocytes and bone marrow-derived mesenchymal stem cells co-cultured extracellular vesicles of the first step and the immune-tolerized trophoblast-derived extracellular vesicles of the second step under temperature change and vibration culture conditions similar to those in the body during pregnancy.

Here, the method includes a fourth step of inoculating the subcultured fetal stem cells into a culture plate, culturing the cells in a serum-free medium, and obtaining a culture supernatant; and a fifth step of performing multi-stage filtration and separation on the culture supernatant.

The extracellular vesicles obtained through the co-culture of human chondrocytes and bone marrow-derived mesenchymal stem cells of the first step are manufactured through step 1-1 of inoculating human chondrocytes and bone marrow-derived mesenchymal stem cells onto the upper and lower parts of a co-culture plate, respectively; step 1-2 of performing the co-culture in a serum-free medium and obtaining a culture supernatant; and step 1-3 of performing multi-stage filtration and separation on the culture supernatant.

The extracellular vesicles obtained through the co-culture of human chondrocytes and bone marrow-derived mesenchymal stem cells in the first step contain collagen types II, VI, IX, and XII, fibronectin, proteoglycan link protein 1 (HPLN-1), extracellular matrix protein-1 (ECM-1), and tissue metalloproteinase inhibitor 1 (TIMP1), which protect the cartilage extracellular matrix and promote the secretion of them from fetal stem cells to maintain cartilage homeostasis.

The extracellular vesicles derived from immune-tolerized trophoblasts that continuously express and secrete HLA-G in the second step are manufactured through step 2-1 of obtaining extracellular vesicles through the co-culture of human amniotic membrane-derived mesenchymal stem cells and amniotic fluid-derived mesenchymal stem cells; step 2-2 of inoculating human trophoblasts into an ex vivo culture matrix gel containing hyaluronic acid and the extracellular vesicles of step 2-1; and step 2-3 of culturing the trophoblasts under temperature change and vibration culture conditions similar to those in the body during pregnancy.

The ex vivo culture matrix gel of the third step contains extracellular vesicles obtained through the co-culture of human amniotic membrane-derived mesenchymal stem cells and amniotic fluid-derived mesenchymal stem cells; and hyaluronic acid.

The ex vivo culture matrix gel of the third step maintains an acidic condition of pH 6 to 7.

The temperature change and vibration culture conditions similar to those in the body during pregnancy in the third step are a temperature change condition having a 5-day cycle in which the temperature changes in a range of 36.0° C. to 37.0° C. and a vibration culture condition having a 24-hour cycle in which the vibration changes in a range of 0 RPM to 60 RPM.

The subcultured fetal stem cells of the fourth step have HLA-G protein present on the cell surface or in the culture supernatant.

The obtained fetal stem cell-derived extracellular vesicles promote the differentiation into hyaline cartilage and reduce joint inflammation.

The pharmaceutical composition for osteoarthritis treatment according to an embodiment of the present invention contains any one of the extracellular vesicles described above.

### Advantageous Effect

According to the present invention, the immune-tolerized fetal stem cell-derived extracellular vesicles contain various components that promote hyaline cartilage regeneration and relieve inflammation at high levels compared to stem cell-derived extracellular vesicles obtained under general culture condition, and thus exhibit superior hyaline cartilage regeneration and anti-inflammatory efficacies.

According to the present invention, the immune-tolerized fetal stem cell-derived extracellular vesicles can deliver active ingredients completely into target cells and tissues without causing immune rejection by allogeneic extracellular vesicles and can thus be used as fundamental disease-modifying osteoarthritis drugs (DMOADs) without side effects.

### BRIEF DESCRIPTION OF DRAWINGS

FIG. 1 is a schematic diagram illustrating a manufacturing process of immune-tolerized fetal stem cell-derived extracellular vesicles for osteoarthritis treatment having hyaline cartilage regeneration and anti-inflammatory efficacies according to an embodiment of the present invention;

FIG. 2 is a graph illustrating the results of analyzing the content of extracellular vesicles contained in immune-tolerized fetal stem cell-derived extracellular vesicles for osteoarthritis treatment (itOA-FSC-EVs) having hyaline cartilage regeneration and anti-inflammatory efficacies according to an embodiment of the present invention by nanoparticle tracking analysis (NTA);

FIG. 3 is a graph illustrating the results of analyzing the content of extracellular vesicles contained in immune-tolerized fetal stem cell-derived extracellular vesicles (itFSC-EVs) according to an embodiment of the present invention by nanoparticle tracking analysis (NTA);

FIG. 4 illustrates the results of analyzing the content of extracellular vesicles contained in human bone marrow mesenchymal stem cell-derived extracellular vesicles (BM-MSC-EVs) according to an embodiment of the present invention by nanoparticle tracking analysis (NTA);

FIG. 5 is a graph illustrating the results of analyzing the expression levels of exosome-specific markers (CD9, CD63, and CD81) in immune-tolerized fetal stem cell-derived extracellular vesicles for osteoarthritis treatment (itOA-FSC-EVs) having hyaline cartilage regeneration and anti-inflammatory efficacies according to an embodiment of the present invention using ExoView equipment;

FIG. 6 is a graph illustrating the results of analyzing the expression levels of exosome-specific markers (CD9, CD63, and CD81) in immune-tolerized fetal stem cell-derived extracellular vesicles (itFSC-EVs) according to an embodiment of the present invention using ExoView equipment;

FIG. 7 is a graph illustrating the results of analyzing the expression levels of exosome-specific markers (CD9, CD63, and CD81) in human bone marrow mesenchymal stem cell-derived extracellular vesicles (BM-MSC-EVs) according to an embodiment of the present invention using ExoView equipment;

FIG. 8 is a graph illustrating the results of analyzing the relative expressions of microRNAs (miRNAs) related to cartilage protection and regeneration, which are contained in immune-tolerized fetal stem cell-derived extracellular vesicles for osteoarthritis treatment (itOA-FSC-EVs) having hyaline cartilage regeneration and anti-inflammatory efficacies, immune-tolerized fetal stem cell-derived extracellular vesicles (itFSC-EVs), and human bone marrow mesenchymal stem cell-derived extracellular vesicles (BM-MSC-EVs) according to an embodiment of the present invention by next generation sequencing (NGS);

FIG. 9 is a graph illustrating the results of analyzing the relative expressions of genes that promote hyaline cartilage regeneration or degrade cartilage extracellular matrix, which are contained in immune-tolerized fetal stem cell-derived extracellular vesicles for osteoarthritis treatment (itOA-FSC-EVs) having hyaline cartilage regeneration and anti-inflammatory efficacies, immune-tolerized fetal stem cell-derived extracellular vesicles (itFSC-EVs), and human bone marrow mesenchymal stem cell-derived extracellular vesicles (BM-MSC-EVs) according to an embodiment of the present invention by next-generation sequencing (NGS);

FIG. 10 is a graph illustrating the results of analyzing the content of an inflammatory cytokine (interleukin-6, IL-6) secreted from macrophages by ELISA when human macrophages stimulated with LPS (lipopolysaccharide) are treated with each of the immune-tolerized fetal stem cell-derived extracellular vesicles for osteoarthritis treatment (itOA-FSC-EVs) having hyaline cartilage regeneration and anti-inflammatory efficacies, immune-tolerized fetal stem cell-derived extracellular vesicles (itFSC-EVs), and human bone marrow mesenchymal stem cell-derived extracellular vesicles (BM-MSC-EVs) according to an embodiment of the present invention at different concentrations;

FIG. 11 is a graph illustrating the results of analyzing the content of an inflammatory cytokine (tumor necrosis factor-α, TNF-α) secreted from macrophages by ELISA when human macrophages stimulated with LPS (lipopolysaccharide) are treated with each of the immune-tolerized fetal stem cell-derived extracellular vesicles for osteoarthritis treatment (itOA-FSC-EVs) having hyaline cartilage regeneration and anti-inflammatory efficacies, immune-tolerized fetal stem cell-derived extracellular vesicles (itFSC-EVs), and human bone marrow mesenchymal stem cell-derived extracellular vesicles (BM-MSC-EVs) according to an embodiment of the present invention at different concentrations;

FIG. 12 is a graph illustrating the results of analyzing the content of an inflammatory cytokine (prostaglandin E2, PGE2) secreted from macrophages by ELISA when human macrophages stimulated with LPS (lipopolysaccharide) are treated with each of the immune-tolerized fetal stem cell-derived extracellular vesicles for osteoarthritis treatment (itOA-FSC-EVs) having hyaline cartilage regeneration and anti-inflammatory efficacies, immune-tolerized fetal stem cell-derived extracellular vesicles (itFSC-EVs), and human bone marrow mesenchymal stem cell-derived extracellular vesicles (BM-MSC-EVs) according to an embodiment of the present invention at different concentrations;

FIG. 13 is a graph illustrating the RT-qPCR analysis results of the gene expression levels of extracellular matrix components (collagen II and aggrecan), cartilage-degrading enzymes (MMP13 and ADAMTP5), and a hyaline cartilage marker (SOX9) in human chondrocytes treated with each of the immune-tolerized fetal stem cell-derived extracellular vesicles for osteoarthritis treatment (itOA-FSC-EVs) having hyaline cartilage regeneration and anti-inflammatory efficacies, immune-tolerized fetal stem cell-derived extracellular vesicles (itFSC-EVs), and human bone marrow mesenchymal stem cell-derived extracellular vesicles (BM-MSC-EVs) according to an embodiment of the present invention;

FIG. 14 is a graph illustrating the Western blot analysis results of the protein expression levels of extracellular matrix components (collagen II and aggrecan), cartilage-degrading enzymes (MMP13 and ADAMTP5), and a hyaline cartilage marker (SOX9) in human chondrocytes treated with each of the immune-tolerized fetal stem cell-derived extracellular vesicles for osteoarthritis treatment (itOA-FSC-EVs) having hyaline cartilage regeneration and anti-inflammatory efficacies, immune-tolerized fetal stem cell-derived extracellular vesicles (itFSC-EVs), and human bone marrow mesenchymal stem cell-derived extracellular vesicles (BM-MSC-EVs) according to an embodiment of the present invention;

FIG. 15 is an image obtained using an optical microscope/digital slide scanner after staining knee joint sample tissue with SFO (Safranin O) 4 weeks and 12 weeks after administration to the joint space of osteoarthritis-induced rats to evaluate and compare the hyaline cartilage regeneration efficacy of immune tolerized fetal stem cell-derived extracellular vesicles for osteoarthritis treatment (itOA-FSC-EVs) having hyaline cartilage regeneration and anti-inflammatory efficacies and immune tolerized fetal stem cell-derived extracellular vesicles (itFSC-EVs) according to one embodiment of the present invention;

FIG. 16 is a graph illustrating the results of OARSI evaluation on knee joint samples 12 weeks after administration to the joint cavity of rats with osteoarthritis to evaluate and compare with the cartilage regeneration efficacies of immune tolerized fetal stem cell-derived extracellular vesicles for treating osteoarthritis (itOA-FSC-EVs) having cartilage regeneration and anti-inflammatory efficacies and immune tolerized fetal stem cell-derived extracellular vesicles (itFSC-EVs) according to one embodiment of the present invention;

FIG. 17 is a graph illustrating the results of histological evaluation through immunohistochemical staining on knee joint samples 12 weeks after administration to the joint space of rats with osteoarthritis to evaluate and compare with hyaline cartilage regeneration efficacies of immune tolerized fetal stem cell-derived extracellular vesicles for treating osteoarthritis (itOA-FSC-EVs) having hyaline cartilage regeneration and anti-inflammatory efficacies and immune tolerized fetal stem cell-derived extracellular vesicles (itFSC-EVs) according to one embodiment of the present invention.

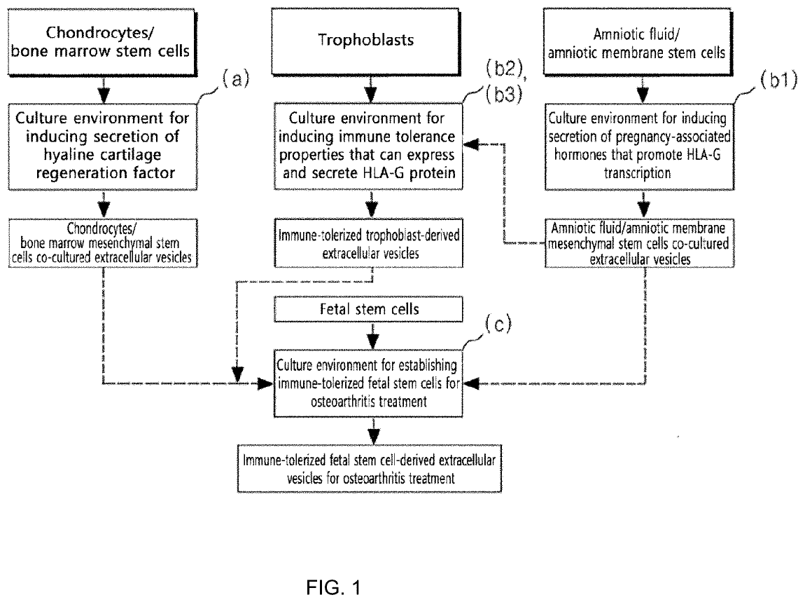

### DETAILED DESCRIPTION OF THE INVENTION

The advantages and features of the present invention and the methods for achieving them will become apparent with reference to the embodiments described below together with the accompanying drawings. However, the present invention is not limited to the embodiments disclosed below and may be implemented in various different forms. These embodiments are provided solely to ensure that the disclosure of the present invention is complete and to fully inform those skilled in the art of the scope of the invention, and the present invention is defined solely by the scope of the claims.

An embodiment of the present invention provides immune-tolerized fetal stem cell-derived extracellular vesicles having hyaline cartilage regeneration and inflammation relief efficacies. The fetal stem cell-derived extracellular vesicles of the present invention are cultured by applying ex vivo culture conditions that simulate the in vivo environment during pregnancy, thereby containing HLA-G protein and exhibiting immune tolerance properties.

The term “extracellular vesicle” of the present invention refers to a vesicle that is produced in a cell and secreted outside the cell, and includes exosomes, microvesicles, microparticles and the like, but is not limited thereto.

In the present invention, the fetal stem cell-derived extracellular vesicles may contain various components having hyaline cartilage regeneration and anti-inflammatory efficacies.

Specifically, the fetal stem cell-derived extracellular vesicles having hyaline cartilage regeneration efficacy may contain FGF-2, FGF-7, IGF-1, TGF-β1, BMP-2, BMP-5, and BMP-7, which induce hyaline cartilage differentiation in osteoarthritic lesions.

The term “fibroblast growth factor-2 (FGF-2)” of the present invention is also called basic fibroblast growth factor (bFGF), and may play a role in stimulating early chondrogenic differentiation by promoting the expression of the SOX9 gene.

The term “fibroblast growth factor-7 (FGF-7)” of the present invention is also called keratinocyte growth factor (KGF), and may play a role in stimulating the development, proliferation, and differentiation of chondrocytes by promoting the expression of connexin 43 (Cx43) and runt-related transcription factor 2 (RUNX2) genes.

The term “insulin-like growth factor-1 (IGF-1)” of the present invention may play a role in promoting the proliferation of chondrocytes, enhancing the production of extracellular matrix, and inhibiting chondrocyte death.

The term “transforming growth factor beta-1 (TGF-β1)” of the present invention may play a role in promoting the initial stage of cartilage formation, that is, the initial differentiation stage of bone marrow-derived mesenchymal stem cells into chondrocytes.

The term “bone morphogenetic protein 4 (BMP-4)” of the present invention may play a role in promoting the differentiation of bone marrow-derived mesenchymal stem cells into chondrocytes and stimulating the synthesis of cartilage matrix components collagen II and aggrecan, thereby promoting cartilage formation.

The term “bone morphogenetic protein 5 (BMP-5)” of the present invention may play a role in promoting the proliferation of chondrocytes and cartilage matrix synthesis.

The term “bone morphogenetic protein 7 (BMP-7)” of the present invention may play a role in promoting cartilage formation by upregulating the metabolism of chondrocytes and protein synthesis.

The extracellular vesicles may additionally contain IL-1ra, IL-4, IL-10, and IL-13, which reduce the inflammatory response.

The term “interleukin-1 receptor antagonist (IL-1ra)” of the present invention refers to an antagonist of a receptor that binds to interleukin-1 (IL-1), an inflammatory cytokine, and may play a role in regulating the inflammatory response and cartilage degeneration by IL-1, particularly by first binding to the IL-1 receptor expressed by chondrocytes and synovial cells.

The term “interleukin-4 (IL-4)” of the present invention is a well-known anti-inflammatory cytokine, and particularly affects proteoglycan metabolism by inhibiting MMP, which degrades cartilage matrix, and may play a role in protecting cartilage by preventing the death of chondrocytes and synovial cells.

The term “interleukin-10 (IL-10)” of the present invention is a well-known anti-inflammatory cytokine, particularly affects proteoglycan metabolism by inhibiting MMP, which degrades cartilage matrix, and may play a role in stimulating the synthesis of collagen II and aggrecan, which are important components of the cartilage extracellular matrix. The interleukin-10 (IL-10) may also play a role in preventing the death of chondrocytes, like IL-4.

The term “interleukin-13 (IL-13)” of the present invention is a well-known anti-inflammatory cytokine, and may play a role in converting M1-type macrophages, a pro-inflammatory phenotype activated particularly by IL-4, into M2-type macrophages, an anti-inflammatory phenotype, thereby producing anti-inflammatory cytokines such as IL-1ra, IL-10, TGF-β1, and tissue repair factors, and resolving the inflammatory response and suppressing osteoarthritis.

The extracellular vesicles may additionally contain TIMP1 and TIMP2, which inhibit matrix metalloproteinases (MMPs), protein-degrading enzymes that degrade the cartilage extracellular matrix and cause osteoarthritis.

The terms “tissue metalloproteinase inhibitor 1 (TIMP1)” and TIMP2 of the present invention may play a role in protecting the joint extracellular matrix by inhibiting various matrix-degrading enzymes, including MMPs, a disintegrin and metalloproteinases (ADAMs), and a disintegrin and metalloproteinases with thrombospondin motif 5 (ADAMTS5).

The extracellular vesicles may additionally contain various microRNAs (miRNAs), including miR-26a, miR-26b, miR-92a, miR-127, miR-136, miR-140, miR-146a, and miR-148, that protect the cartilage extracellular matrix and maintain homeostasis by inhibiting proteolytic enzymes that degrade the cartilage extracellular matrix.

The term “microRNA (miRNA)” of the present invention refers to a short non-coding RNA molecule composed of about 22 nucleotides found in plants, animals, and microorganisms, and may play a role in regulating gene expression by binding to complementary base pairs in an mRNA molecule.

The term “miR-26a” of the present invention is a microRNA that regulates the expression of the karyopherin subunit alpha 3 (KPNA3) and nitric oxide synthase 2 (NOS2) genes, and KPNA in osteoarthritic cartilage binds to nuclear factor-kappa B (NF-κB) 3 and induces the production of inflammatory cytokines cyclooxygenase-2 (COX-2) and MMP, thereby causing joint inflammation and degeneration of articular cartilage. Since the activation of NOS2 causes chondrocyte death, cartilage degradation, and inhibition of matrix synthesis via overproduction of nitric oxide (NO), upregulation of miR-26a may play a role in protecting cartilage in osteoarthritis.

The term “miR-26b” of the present invention is a microRNA that regulates the expression of the KPNA3 gene. In osteoarthritic cartilage, KPNA3 binds to NF-κB and induces the production of inflammatory cytokines COX-2 and MMP, thereby causing joint inflammation and degeneration of articular cartilage, and upregulation of miR-26b may play a role in protecting cartilage in osteoarthritis.

The term “miR-92a” of the present invention is a microRNA that regulates the expression of the Wnt5A gene. Since Wnt5A plays a key role in cartilage destruction and cartilage matrix degradation by activating MMPs in the pathogenesis of osteoarthritis, upregulation of miR-92a may play a role in protecting cartilage in osteoarthritis, including maintaining cartilage homeostasis, enhancing cartilage development, and preventing cartilage degradation.

The term “miR-127” of the present invention is a microRNA that regulates the expression of the CDH11 gene. Since cadherin-11 (CDH11) activates the Wnt/β-catenin pathway, which inhibits the proliferation of chondrocytes and induces their death, upregulation of miR-127 may play a role in alleviating osteoarthritis by promoting the proliferation of chondrocytes and inhibiting their death.

The term “miR-136” of the present invention is a microRNA that regulates the expression of the E74-like factor 3 (ELF3) gene. Since ELF3 induces cartilage degeneration by inhibiting the proliferation and migration of chondrocytes and the secretion of cartilage matrix, upregulation of miR-136 may promote the proliferation and migration of chondrocytes and inhibit cartilage degeneration in osteoarthritis by increasing the expression of collagen II, aggrecan, and SOX9.

The term “miR-140” of the present invention is a microRNA that regulates the expression of ADAMTS5, MMP13, insulin-like growth factor binding protein 5 (IGFBP5), and RAS like proto-oncogene A (RALA) genes. ADAMTS5 and MMP13 are proteolytic enzymes that mediate the degradation of various components that make up the cartilage matrix and may play an important role in the onset of osteoarthritis. In addition, IGFBP5 may play a role in the pathology of osteoarthritis by regulating the availability of IGF-1 in the joint by binding to IGF-1, which promotes chondrocyte differentiation. RALA is a nucleotide guanosine triphosphate hydrolase enzyme (GTPase) that is a key regulator of cartilage development and downregulates the expression of the SOX9 gene, which enhances the production of cartilage matrix components, and upregulation of miR-140 may play a role in protecting cartilage and suppressing the onset of osteoarthritis.

The term “miR-146a” of the present invention is a microRNA that regulates the expression of the MMP13 gene, and may play a role in protecting osteoarthritic cartilage by inhibiting the production of MMP13 and ADAMTS5 induced by IL-1β, an inflammatory cytokine, in osteoarthritic lesions, the synthesis of collagen II and aggrecan, which are the cartilage matrix components, and the upregulation of TNF-α.

The term “miR-148” of the present invention is a microRNA that regulates the expression of ADAMTS and MMP genes. Since ADAMTS and MMP are proteolytic enzymes that mediate the decomposition of various components that make up the cartilage matrix and thus may play an important role in the onset of osteoarthritis, upregulation of miR-148 may play a role in protecting cartilage by inhibiting the degradation of cartilage matrix.

The extracellular vesicles may additionally contain aggrecan, collagen II, proteoglycan 4 (PRG4), and SOX9, which play a role in maintaining cartilage homeostasis by promoting synthesis and protecting the cartilage extracellular matrix from various causes, including inflammation.

The term “aggrecan” of the present invention refers to a type of proteoglycan, a major extracellular matrix molecule, and may play a role in making up and protecting articular cartilage.

The term “collagen II” of the present invention is a main component that constitutes the core of the collagen fiber structure of articular cartilage.

The term “sex-determining region Y (SRY)-related protein 9 (SOX9)” of the present invention is a hyaline cartilage marker mainly expressed in differentiating and proliferating chondrocytes, and may play a role in maintaining the homeostasis of the cartilage extracellular matrix.

The term “proteoglycan 4 (PRG4)” of the present invention is an extracellular matrix protein secreted by synovial fibroblasts and surface layer chondrocytes, and may play a role in regulating joint homeostasis.

The extracellular vesicles may additionally contain progesterone, HLA-G1, HLA-G2, HLA-G5, and HLA-G6.

The term “HLA-G” of the present invention refers to a protein called human leukocyte antigen G or HLA-G histocompatibility antigen class, G, and the like. HLA-G was first discovered in extravillous trophoblasts (EVTs) present at the maternal-fetal interface during pregnancy, and exists as a heterologous form that is expressed only on the cell membrane (membrane-bound HLA-G, including HLA-G1, G2, G3, and G4) or a soluble form that can be secreted outside the cell in the form of a single molecule (soluble HLA-G, including HLA-G5, G6, and G7) by alternative splicing of HLA-G mRNAs.

In the present invention, HLA-G protein reduces the cytotoxicity of immune cells and promotes the differentiation of regulatory T cells due to its low polymorphism and specificity for acting on immune cells, and thus plays an essential role in establishing an immune tolerance environment that protects the fetus, a semi-allograft, from the maternal immune system, especially during pregnancy.

Still another embodiment of the present invention provides a method for manufacturing immune-tolerized fetal stem cell-derived extracellular vesicles for osteoarthritis treatment, which includes step (a) of obtaining extracellular vesicles containing the cartilage extracellular matrix through the co-culture of human chondrocytes and bone marrow-derived mesenchymal stem cells; step (b) of obtaining the extracellular vesicles derived from immune-tolerized trophoblasts that continuously express and secrete HLA-G; and step (c) of inoculating fetal stem cells into an ex vivo culture matrix gel and subculturing the cells in a serum-free medium containing the human chondrocytes and bone marrow-derived mesenchymal stem cells co-cultured extracellular vesicles of step (a) and the immune-tolerized trophoblast-derived extracellular vesicles of step (b) under temperature change and vibration culture conditions similar to those in the body during pregnancy.

The term “trophoblast” of the present invention refers to a type of cell that forms the placenta, and may refer to a cell that provides signal transmission and nutrients related to embryonic development to the inner cell mass in the early stage of development, induces successful implantation by creating an immune tolerance environment that protects the fertilized embryo from the mother's immune system during the early stage of implantation, and thereafter plays an important role in the maintenance of pregnancy and development of the fetus by forming the placenta and continuously expressing and secreting the HLA-G protein.

During pregnancy, the fetus exists in amniotic fluid surrounded by the amniotic membrane, and the fetal membrane is composed of the amnion and the chorion containing trophoblasts, and the placenta is composed of the fetal chorion and the maternal decidual.

The present inventors have developed an ex vivo culture matrix gel for trophoblasts containing human amniotic fluid and amniotic membrane stem cell-derived extracellular vesicles to simulate the fetal membrane structure during pregnancy in vitro through a prior patent, and applied the ex vivo culture matrix gel to this patent to obtain immune-tolerized trophoblast-derived extracellular vesicles (U.S. Pat. No. 11,566,220 B2 and U.S. Pat. No. 11,771,720 B2).

In this way, the immune-tolerized trophoblast-derived extracellular vesicles and extracellular vesicles containing the cartilage extracellular matrix, which are obtained by co-culturing human chondrocytes and bone marrow stem cells to induce hyaline cartilage regeneration factors are applied to an ex vivo culture condition simulating the in vivo environment during pregnancy to manufacture immune-tolerized fetal stem cell-derived extracellular vesicles having hyaline cartilage regeneration and anti-inflammatory efficacies.

The method for manufacturing immune-tolerized fetal stem cell-derived extracellular vesicles having hyaline cartilage regeneration and anti-inflammatory efficacies may additionally include step (d) of inoculating the subcultured fetal stem cells into a culture plate, culturing the cells in a serum-free medium, and obtaining a culture supernatant; and step (e) of performing multi-stage filtration and separation on the culture supernatant.

In the manufacturing method, the extracellular vesicles obtained through the co-culture of human chondrocytes and bone marrow-derived mesenchymal stem cells in step (a) may be manufactured through step (a-1) of inoculating human chondrocytes and bone marrow-derived mesenchymal stem cells onto the upper and lower parts of a co-culture plate, respectively; step (a-2) of performing the co-culture in a serum-free medium and obtaining a culture supernatant; and step (a-3) of performing multi-stage filtration and separation on the culture supernatant.

Specifically, step (a-1) may include step (a-1-1) of cryopreserving human chondrocytes and bone marrow-derived mesenchymal stem cells in a cryopreservation composition containing immune-tolerized human mesenchymal stem cell-derived extracellular vesicles at 5% to 50% (v/v); and step (a-1-2) of thawing the cryopreserved human chondrocytes and bone marrow-derived mesenchymal stem cells, inoculating the cells onto the lower and upper parts of a co-culture plate, respectively, but is not limited thereto.

Specifically, in step (a-1-2), human chondrocytes and bone marrow-derived mesenchymal stem cells may be inoculated at a density of 5,000 to 20,000 cells/cm², respectively, but the inoculation density is not limited thereto.

The co-culture in step (a-1) may be performed for 100 to 140 hours, specifically 114 to 126 hours, but is not limited thereto.

In step (a-3), the culture supernatant may be separated 1 to 10 times, preferably 4 times using a filter of 0.3 μm to 1 μm, specifically 0.45 μm to 0.8 μm. In a specific example, the culture supernatant may be separated two times 0.8 μm and 0.45 μm filters, respectively, but is not limited thereto.

In the manufacturing method, the extracellular vesicles obtained through co-culturing human chondrocytes and bone marrow-derived mesenchymal stem cells in step (a) may contain various components that induce secretion of cartilage regeneration factors in fetal stem cells.

In the manufacturing method, the extracellular vesicles derived from immune-tolerized trophoblasts that express and secrete HLA-G in step (b) may be manufactured through step (b-1) of obtaining extracellular vesicles through the co-culture of human amniotic membrane-derived mesenchymal stem cells and amniotic fluid-derived mesenchymal stem cells; step (b-2) of inoculating human trophoblasts into an ex vivo culture matrix gel containing hyaluronic acid and the extracellular vesicles of step (b-1); and step (b-3) of culturing the trophoblasts under temperature change and vibration culture conditions similar to those in the body during pregnancy.

Specifically, step (b-1) may include step (b-1-1) of cryopreserving human amniotic membrane and amniotic fluid-derived mesenchymal stem cells in a cryopreservation composition containing immune-tolerized human mesenchymal stem cell-derived extracellular vesicles at 5% to 50% (v/v); step (b-1-2) of thawing the cryopreserved human amniotic membrane and amniotic fluid-derived mesenchymal stem cells, inoculating the cells onto the lower and upper parts of a co-culture plate, respectively, co-culturing the cells in a serum-free medium, and obtaining a culture supernatant, but is not limited thereto.

In step (b-1), the extracellular vesicles and hyaluronic acid may be mixed at a weight ratio of 1:1 to 1:20 to manufacture the ex vivo culture matrix gel.

In step (b-1), trophoblasts may be inoculated at a density of 5,000 to 15,000 cells/cm².

Specifically, steps (b-2) and (b-3) may be steps of inoculating human trophoblasts into an ex vivo culture matrix gel manufactured by mixing the extracellular vesicles obtained by multi-stage filtration and separation of the culture supernatant and hyaluronic acid and subculturing the cells in a serum-free medium, but are not limited thereto.

In step (b-3), the subcultured trophoblasts may be washed, inoculated onto a general culture plate at a density of 10,000 to 30,000 cells/cm², and then subjected to serum-free culture for 72 to 120 hours, and then the culture supernatant may be obtained.

The trophoblast-derived extracellular vesicles may be manufactured through step (b-4) of inoculating the subcultured trophoblasts onto a culture plate, then performing serum-free culture, and performing multi-stage filtration and separation on the obtained culture supernatant in addition to the above steps.

At this time, the culture supernatant may be separated 1 to 10 times, preferably 4 times using a filter of 0.3 μm to 1 μm, specifically 0.45 μm to 0.8 μm. In a specific example, the culture supernatant may be separated two times using 0.8 μm and 0.45 μm filters, respectively, but is not limited thereto.

The extracellular vesicles derived from immune-tolerized trophoblasts that secrete and express HLA-G, obtained by the method of step (b) may contain HLA-G1, HLA-G2, HLA-G5, and HLA-G6 proteins, which induce an immune tolerance environment, and progesterone, which promotes mRNA expression of the HLA-G gene.

The ex vivo culture matrix gel of step (c) may contain extracellular vesicles obtained through the co-culture of human amniotic membrane-derived mesenchymal stem cells and amniotic fluid-derived mesenchymal stem cells; and hyaluronic acid.

Specifically, by adding hyaluronic acid, the ex vivo culture matrix gel can maintain an acidic condition of pH 6 to 7.

The temperature change and vibration culture conditions similar to those in the body during pregnancy in step (c) may be a temperature change condition based on the basal body temperature method having a 5-day cycle in which the temperature changes in a range of 36.0° C. to 37.0° C. and a vibration culture condition having a 24-hour cycle in which the vibration changes in a range of 0 RPM to 60 RPM.

Specifically, the temperature change condition may be, but is not limited to, a temperature change condition in which the temperature changes to 36.5° C. at the 0 to 12th hour, 36.4° C. at the 12th to 36th hour, 36.3° C. at the 36th to 48th hour, 36.2° C. at the 48th to 60th hour, 36.0° C. at the 60th to 72nd hour, and to 37.0° C. at the 72nd to 120th hour.

Specifically, the vibration culture condition may be, but is not limited to, a vibration culture condition in which the vibration changes to 0 RPM at the 0 to 7th hour, 30 RPM at the 8th hour, 60 RPM at the 9th to 18th hour, 20 RPM at the 19th hour, and 0 RPM at the 20th to 24th hour.

The immune-tolerized fetal stem cell-derived extracellular vesicles obtained through the above manufacturing method may contain various components having hyaline cartilage regeneration and anti-inflammatory efficacies.

Still another embodiment of the present invention provides a pharmaceutical composition for osteoarthritis treatment, which contains the immune-tolerized fetal stem cell-derived extracellular vesicles containing various components having hyaline cartilage regeneration and anti-inflammatory efficacies.

In the Examples of the present invention, when chondrocytes were treated with the fetal stem cell-derived extracellular vesicles, the expression of various components, which promote cartilage regeneration, inhibit cartilage degradation, and protect cartilage, significantly increased. When fetal stem cell-derived extracellular vesicles were administered to an osteoarthritis-induced animal model, excellent hyaline cartilage regeneration efficacy was found, and it was verified that the extracellular vesicles can be used for the treatment of osteoarthritis.

The term “osteoarthritis” of the present invention refers to a disease caused by damage to articular cartilage and underlying tibial tissue. Although there is also rheumatoid arthritis, osteoarthritis is the most common form of arthritis. Symptoms include joint pain and stiffness, and at first, pain is felt only when moving, but if it becomes chronic, pain becomes constant. Osteoarthritis becomes more common with age and affects millions of people worldwide. Osteoarthritis is a typical degenerative disease that continues to progress once it occurs. Common early symptoms include difficulty moving the joint and pain. Any joint can be affected by osteoarthritis, but osteoarthritis is most commonly seen in the fingers, neck, lower spine, knees, and hips.

Specifically, osteoarthritis may involve, but is not limited to, the joints of the fingers, neck, lower spine, knees, and hips.

The term “treatment” of the present invention means any action by which symptoms of osteoarthritis are improved or beneficially changed by the composition.

In the present invention, the composition is preferably used for humans, but may also be used for livestock such as cows, horses, sheep, pigs, goats, camels, antelopes, dogs or cats that may develop osteoarthritis.

In the pharmaceutical composition for osteoarthritis treatment of the present invention, the route and method of administration are not particularly limited, and any route and method of administration may be adopted as long as the composition can reach the intended site.

Specifically, the composition may be administered through various routes, either orally or parenterally. Non-limiting examples of the route of administration include ocular, oral, rectal, topical, intravenous, intraperitoneal, intramuscular, intraarterial, transdermal, or intranasal administration or administration through inhalation. The composition may be administered by any device capable of transporting the active substance to the target cell.

In the present invention, the pharmaceutical composition for osteoarthritis treatment may additionally contain a pharmaceutically acceptable carrier, excipient or diluent commonly used in the manufacture of pharmaceutical compositions, and the carrier may include a non-naturally occurring carrier.

In the present invention, the term “pharmaceutically acceptable” means that the composition exhibits the property of being non-toxic to cells or humans exposed to the composition.

More specifically, the pharmaceutical composition may be formulated and used in the form of oral preparations such as powders, granules, tablets, capsules, suspensions, emulsions, syrups, and aerosols, external preparations, suppositories, and sterile injectable solutions according to conventional methods, but is not limited thereto as long as it is a formulation used for the treatment of osteoarthritis in the art.

Specific examples of the carriers, excipients, and diluents that may be contained in the pharmaceutical composition include lactose, dextrose, dextran, sucrose, sorbitol, mannitol, xylitol, erythritol, maltitol, starch, acacia gum, alginate, gelatin, calcium phosphate, calcium silicate, cellulose, methyl cellulose, microcrystalline cellulose, polyvinyl pyrrolidone, water, methyl hydroxybenzoate, propyl hydroxybenzoate, talc, magnesium stearate, polycaprolactone (PCL), polylactic acid (PLA), poly-L-lactic acid (PLLA), and mineral oil.

In a case where the pharmaceutical composition is formulated into a preparation, it may be prepared using diluents or excipients such as fillers, bulking agents, binders, wetting agents, disintegrants, and surfactants that are commonly used.

Solid preparations for oral administration include tablets, pills, powders, granules, and capsules, and these solid preparations may be prepared by mixing the extract and its fractions with at least one or more excipients, for example, starch, calcium carbonate, sucrose or lactose, and gelatin. In addition to simple excipients, lubricants such as magnesium stearate and talc may also be used.

Liquid preparations for oral administration include suspensions, oral liquids, emulsions, and syrups, and may contain various excipients, such as wetting agents, sweeteners, flavoring agents, and preservatives in addition to the commonly used simple diluents, such as water and liquid paraffin.

Preparations for parenteral administration may include sterile aqueous solutions, non-aqueous solutions, suspensions, emulsions, lyophilized preparations, and suppositories.

As non-aqueous solvents and suspending agents, propylene glycol, polyethylene glycol, vegetable oils such as olive oil, injectable esters such as ethyl oleate, and the like may be used.

As suppository bases, Witepsol, macrogol, Tween 61, cocoa butter, laurin butter, glycerogelatin, and the like may be used. Polymeric substances such as polycaprolactone (PCL), polylactic acid (PLA), poly-L-lactic acid (PLLA), hyaluronic acid, and dextran may be used in the preparations for intra-articular administration.

Still another aspect of the present invention provides a method for preventing or treating osteoarthritis, which includes administering a pharmaceutical composition containing the immune-tolerized fetal stem cell-derived extracellular vesicles.

The terms osteoarthritis, immune-tolerized fetal stem cell-derived extracellular vesicles, and treatment are as described above.

The term “prevention” of the present invention means any action that suppresses an osteoarthritis disease or delays the onset through the administration of the composition. For the purpose of the present invention, the prevention may be understood as an action that suppresses or delays the onset of an osteoarthritis disease by using the pharmaceutical composition of the present invention, but is not particularly limited thereto.

The term “improvement” of the present invention means any action that at least reduces the degree of osteoarthritis disease.

The term “administration” in the present invention means introducing or treating the pharmaceutical composition of the present invention within a subject by an appropriate method.

The pharmaceutical composition of the present invention may be administered in a pharmaceutically effective amount, and the pharmaceutically effective amount means an amount that is sufficient to treat a disease with a reasonable benefit/risk ratio applicable to medical treatment and does not cause any side effects, and may be readily determined by those skilled in the art based on factors well known in the medical field. The route and method for administering the pharmaceutical composition of the present invention are not particularly limited, and any route and method of administration may be adopted if the composition can reach the subject to achieve the purpose of the present invention.

The “subject” refers to all animals, including humans, that have developed or are capable of developing an osteoarthritis disease.

Still another aspect of the present invention provides a composition for a functional food, a quasi-drug, or a medical device for treating, preventing, alleviating, or improving osteoarthritis, which contains the immune-tolerized fetal stem cell-derived extracellular vesicles.

The term “functional food” of the present invention is the same as food for special health use (FoSHU), and refers to a food with high medical and therapeutic effects that is processed to efficiently exhibit a bioregulatory function in addition to nutritional supply. In the present invention, the functional food is used together with a health food or a health supplement.

The term “quasi-drug” of the present invention refers to articles that have a milder effect than drugs among articles that are used for the purpose of diagnosing, treating, improving, alleviating, curing, or preventing diseases in humans or animals. For example, quasi-drugs under the Pharmaceutical Affairs Act are products other than articles used for pharmaceutical purposes, and include products used to treat or prevent diseases in humans/animals and products that have a mild effect on the human body or do not have a direct effect.

The term “medical device” of the present invention refers to an instrument, machine, device, material, or similar product used alone or in combination for humans or animals, and may include products used (i) for the purpose of diagnosing, treating, alleviating, curing, or preventing diseases; (ii) for the purpose of diagnosing, treating, alleviating, or correcting injuries and disabilities; and (iii) for the purpose of examining, replacing, or modifying structures or functions, but excludes drugs and quasi-drugs under the Pharmaceutical Affairs Act.

Still another embodiment of the present invention provides the use of immune-tolerized fetal stem cell-derived extracellular vesicles for osteoarthritis treatment.

Still another aspect of the present invention provides the use of immune-tolerized fetal stem cell-derived extracellular vesicles as a pharmaceutical composition and a composition for a functional food, a quasi-drug, or a medical device for treating, preventing, alleviating, or improving osteoarthritis.

### Example 1

### Obtaining Human Chondrocytes and Bone Marrow-Derived Mesenchymal Stem Cell Co-Cultured Extracellular Vesicles (HChD/BM-MSC^{CO}-EVs) Containing the Cartilage Extracellular Matrix

A co-culture system of human chondrocytes and bone marrow-derived mesenchymal stem cells was developed to obtain extracellular vesicles containing the cartilage extracellular matrix that induces the secretion of components that promote hyaline cartilage regeneration.

More specifically, in order to exclude foreign antigens that might be contained in the extracellular vesicles, human chondrocytes (PromoCell, Cat. #C-12710) and human bone marrow-derived mesenchymal stem cells (PromoCell, Cat. #C-12974) were stored in a cryopreservation agent (CRYOGUARD®, Stemmedicare) that does not contain fetal bovine serum (FBS) and dimethyl sulfoxide (DMSO) at −80° C. in an ultra-low temperature freezer for 2 weeks.

After each of the stored cells were thawed, for the co-culture, chondrocytes were dispensed into serum-free DMEM/F-12 medium in the lower plate of a multi-plate dish (ThermoFisher, Cat. #140663) at a density of 10,000 cells/cm², and bone marrow-derived stem cells were dispensed into serum-free DMEM/F-12 medium in the upper insert at a density of 20,000 cells/cm², and the co-culture was performed for 96 hours in an incubator at 37° C. and 5% CO₂.

Then, the obtained culture supernatant was centrifuged to remove cells and impurities, and vacuum filtrations were performed two times using 0.8 μm and 0.45 μm filters, respectively, to obtain human chondrocytes and bone marrow-derived mesenchymal stem cells co-cultured extracellular vesicles (HChD/BM-MSC^{CO}-EVs).

In order to compare the content of cartilage extracellular matrix in extracellular vesicles obtained through the co-culture of human chondrocytes and bone marrow-derived mesenchymal stem cells (HChD/BM-MSC^{CO}-EVs) with that in extracellular vesicle groups obtained by singly culturing human chondrocytes (HChD-EVs) and human bone marrow-derived mesenchymal stem cells (BM-MSC-EVs), respectively, LC-MS/MS analysis was conducted, and as a result, it was found that collagen types II, VI, IX, and XII, fibronectin, proteoglycan link protein 1 (HPLN-1), and the extracellular matrix protein-1 (ECM-1), which are the most abundant the extracellular matrix proteins secreted in normal cartilage, were contained at significantly higher levels in HChD/BM-MSC^{CO}-EVs than in HChD-EVs and BM-MSC-EVs (Table 1).

It was also found that TIMP1, an enzyme that inhibits MMP, an enzyme that degrades the extracellular matrix, was contained at significantly higher levels in HChD/BM-MSC^{CO}-EVs than in HChD-EVs and BM-MSC-EVs (Table 1).

TABLE 1

| Extracellular | HChD-EVs | BM-MSC-EVs | HChD/BM-MSC^{CO}- |
|---|---|---|---|
| matrix proteins | (ng/ml) | (ng/ml) | EVs (ng/ml) |
| Collagen II | 5,428 | 1,796 | 9,323 |
| Collagen IX | 450 | 77 | 1,605 |
| Collagen VI | 167 | 76 | 874 |
| Collagen XII | 808 | 120 | 1,471 |
| Fibronectin | 653 | 155 | 1,554 |
| Proteoglycan link | 13.4 | 6.4 | 190.3 |
| protein 1 | | | |
| (HPLN-1) | | | |
| ECM-1 | 98.2 | 28.1 | 228.7 |
| TIMP-1 | 23.0 | 69.9 | 190.1 |

<Comparison of concentrations of major cartilage extracellular matrix proteins in extracellular vesicles obtained through the co-culture of human chondrocytes and bone marrow-derived mesenchymal stem cells (HChD/BM-MSC^{CO}-EVs) with that in extracellular vesicles obtained by singly culturing human chondrocytes (HChD-EVs) and human bone marrow-derived mesenchymal stem cells (BM-MSC-EVs), respectively>

### Example 2

Manufacturing Ex Vivo Culture Matrix for Trophoblasts to Induce Immune Tolerance Properties and Obtaining Immune-Tolerized Trophoblast-Derived Extracellular Vesicles (itTBC-EVs)

A matrix gel for ex vivo culture of trophoblasts capable of inducing immune tolerance properties was manufactured.

More specifically, in order to exclude foreign antigens that might be contained in extracellular vesicles, human amniotic membrane-derived mesenchymal stem cells (ScienCell, Cat. #7140) and mesenchymal stem cells established from amniotic fluid donated from healthy mothers were stored in a cryopreservation agent (MBTC-CRYOGUARD®, Stemmedicare) that does not contain fetal bovine serum (FBS) and dimethyl sulfoxide (DMSO) at −80° C. in an ultra-low temperature freezer for 2 weeks.

After each of the stored cells were thawed, for the co-culture, amniotic membrane-derived mesenchymal stem cells were dispensed into serum-free DMEM/F-12 medium in the lower plate of a multi-plate dish (ThermoFisher, Cat. #140663) at a density of 20,000 cells/cm² and amniotic fluid-derived mesenchymal stem cells were dispensed into serum-free DMEM/F-12 medium in the upper insert at a density of 20,000 cells/cm², and the co-culture was performed for 96 hours in an incubator at 37° C. and 5% CO₂. Then, the obtained culture supernatant was centrifuged to remove cells and impurities.

Vacuum filtrations were performed two times using 0.8 μm and 0.45 μm filters, respectively, to obtain human amniotic fluid and amniotic membrane-derived mesenchymal stem cell co-cultured extracellular vesicles (AF/AM-MSC^{CO}-EVs).

The ex vivo culture matrix gel was manufactured by adding hyaluronic acid powder to the human amniotic fluid and amniotic membrane-derived mesenchymal stem cell co-cultured extracellular vesicles obtained through the method above so that the pH was between 6.6 and 6.8, then thoroughly mixed by vortexing. The gel was dispensed onto a culture plate and evenly spread by shaking the culture plate on a vibrator, into which trophoblasts were dispensed, and subculture was performed in serum-free DMEM/F-12K medium at 5 to 6 day intervals.

To induce progesterone hormone secretion from trophoblasts, a temperature change condition was applied, having a 5-day cycle in the range of 36.0° C. to 37.0° C., similar to the body temperature change of women before and after ovulation, and a vibration culture condition was applied, having a 24-hour cycle in which the vibration varied in the range of 0 RPM to 60 RPM.

In order to examine whether trophoblasts cultured in the ex vivo culture matrix gel could continuously secrete HLA-G protein, while the subculture was performed, the concentration of soluble HLA-G (sHLA-G) protein in the culture supernatant obtained during each subculture was measured using a Human HLA-G ELISA kit (LSBio) using MEM-G/9 antibody.

When the sHLA-G concentration in the culture supernatant exceeded 20 μg/ml for three consecutive passages or more, it was considered that immune-tolerized trophoblasts (itTBCs) were established, and the subculture was stopped.

The immune-tolerized trophoblasts (itTBCs) cultured in the ex vivo culture matrix gel were dispensed at a density of 15,000 cells/cm² into serum-free DMEM/F-12K medium using a general culture plate, and cultured for 96 hours in an incubator at 37° C. and 5% CO₂. Then, the obtained culture supernatant was centrifuged to remove cells and impurities.

Vacuum filtrations were performed two times using 0.8 μm and 0.45 μm filters, respectively, to obtain immune-tolerized trophoblast-derived extracellular vesicles (itTBC-EVs), which contained HLA-G protein and progesterone hormone and could induce immune tolerance properties.

### Example 3

### Manufacturing Extracellular Vesicles for Osteoarthritis Treatment Having Hyaline Cartilage Regeneration and Anti-Inflammatory Efficacies

It was attempted to manufacture extracellular vesicles for osteoarthritis treatment having hyaline cartilage regeneration and anti-inflammatory efficacies.

More specifically, to induce the secretion of extracellular vesicles containing various components having hyaline cartilage regeneration and anti-inflammatory efficacies from target stem cells, fetal stem cells (hFSCs) isolated from amniotic fluid were inoculated into the culture plate containing the ex vivo culture matrix gel to induce immune tolerance properties, which was manufactured in Example 2.

The cells were cultured in a serum-free DMEM/F-12 medium containing the human chondrocytes and bone marrow-derived mesenchymal stem cells co-cultured extracellular vesicles (HChD/BM-MSC^{CO}-EVs) of Example 1 at 10% v/v and in a serum-free DMEM/F-12 medium containing the immune-tolerized trophoblast-derived extracellular vesicles (itTBC-EVs) that induce immune tolerance properties, which could continuously secrete and express HLA-G protein, of Example 2 at 10% v/v.

In order to induce immune tolerance properties, the cells were subcultured in an incubator at 37° C. and 5% CO₂ by applying a temperature change condition having a 5-day cycle and a vibration condition the same as those in Example 2.

In order to examine whether fetal stem cells cultured in the ex vivo culture matrix gel that induces immune tolerance properties could continuously secrete HLA-G protein, while the subculture was performed, the concentration of soluble HLA-G (sHLA-G) protein in the culture supernatant obtained during each subculture was measured using a Human HLA-G ELISA kit (LSBio) using MEM-G/9 antibody.

When the sHLA-G concentration in the culture supernatant exceeded 20 μg/ml for three consecutive passages or more, it was considered that immune-tolerized fetal stem cells for chondrocyte regeneration (itOA-FSCs) for producing extracellular vesicles for osteoarthritis treatment were established, and the subculture was stopped.

The itOA-FSCs established in the ex vivo culture matrix gel as described above were washed with PBS, inoculated onto a general plate not containing the ex vivo culture matrix gel at a density of 2.0×10⁴ cells/cm², and cultured in a serum-free DMEM/F-12 medium for 96 hours in an incubator at 37° C. and 5% CO₂.

The collected culture supernatant was centrifuged to remove cells and impurities, and vacuum filtrations were performed two times using 0.8 μm and 0.45 μm filters, respectively, to obtain immune-tolerized fetal stem cell-derived extracellular vesicles for chondrocyte regeneration (itOA-FSC-EVs) having hyaline cartilage regeneration and anti-inflammatory efficacies.

### Example 4

Characterization of Extracellular Vesicles (itOA-FSC-EVs) for Osteoarthritis Treatment Having Hyaline Cartilage Regeneration and Anti-Inflammatory Efficacies

The characterization of extracellular vesicles for osteoarthritis treatment (itOA-FSC-EVs) having hyaline cartilage regeneration and anti-inflammatory efficacies was performed. As control groups, (i) immune-tolerized fetal stem cell-derived extracellular vesicles (itFSC-EVs) that were cultured in an ex vivo culture matrix gel not containing the human chondrocytes and bone marrow-derived mesenchymal stem cells co-cultured extracellular vesicles (HChD/BM-MSC^{CO}-EVs) of Example 1, so only immune tolerance properties were induced; and (ii) human bone marrow mesenchymal stem cell-derived extracellular vesicles (BM-MSC-EVs) cultured in a general culture plate were used for comparison.
1) Nanoparticle tracking analysis (NTA)

The size and concentration of extracellular vesicles were analyzed using the NS300 (Malvern Panalytical), a nanoparticle tracking analysis (NTA) equipment that could quantitatively measure the size and concentration of extracellular vesicles.

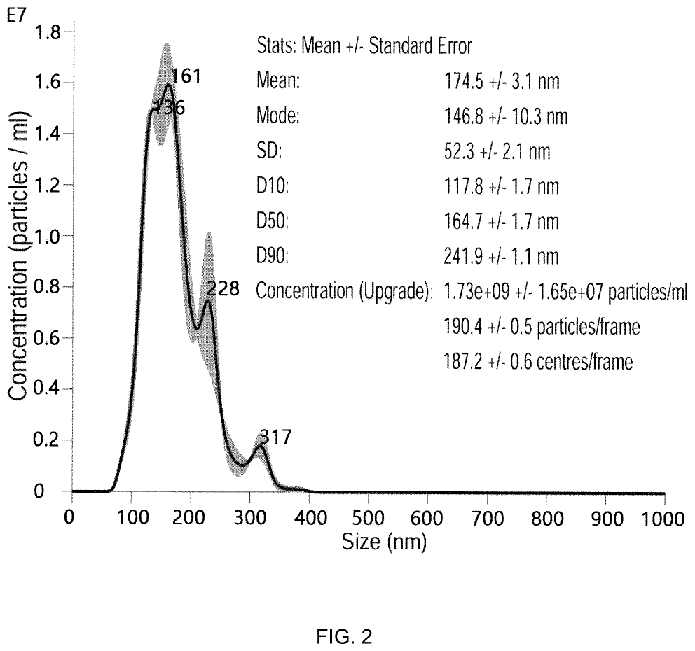

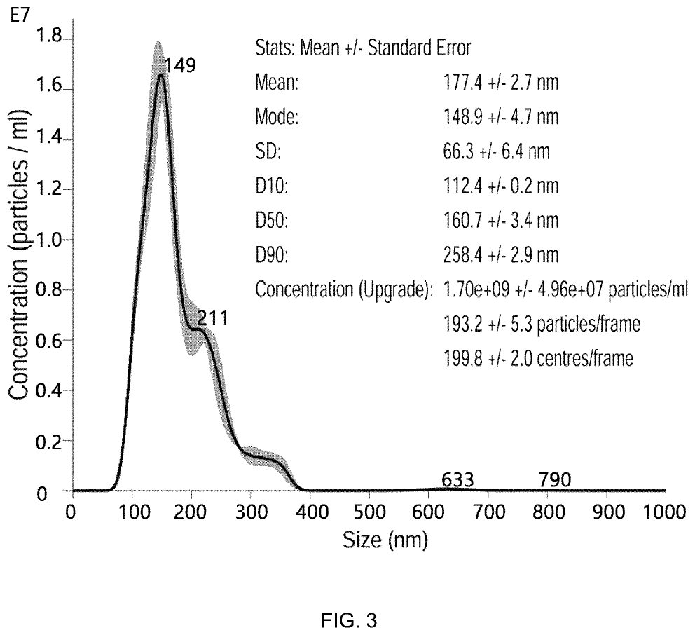

The extracellular vesicles for osteoarthritis treatment (itOA-FSC-EVs) having hyaline cartilage regeneration and anti-inflammatory efficacies had an average size of 174.5 nm and an average concentration of 1.73×10⁹ particles/ml (FIG. 2), and had a similar average size (177.4 nm) and average concentration (1.73×10⁹ particles/ml) distribution to that of a control group, the immune-tolerized fetal stem cell-derived extracellular vesicles (itFSC-EVs) (FIG. 3).

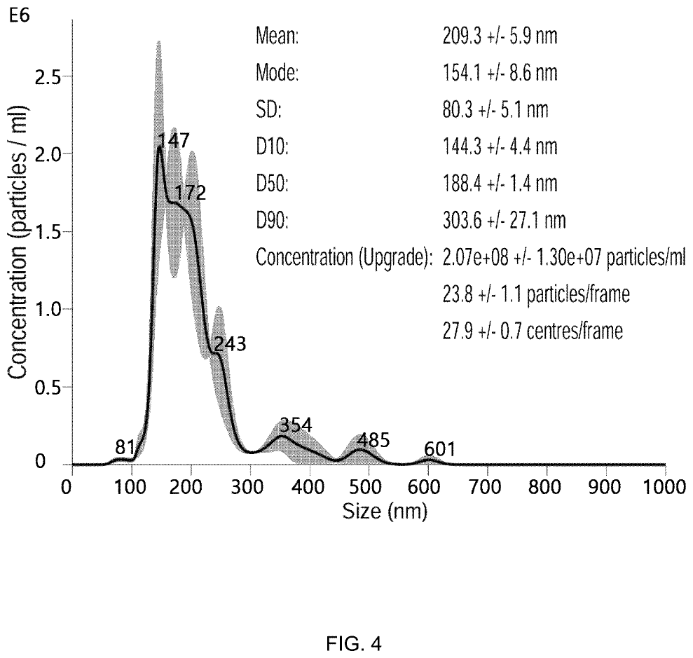

However, it was found that another control group, the bone marrow mesenchymal stem cell-derived extracellular vesicles (BM-MSC-EVs), had an average size of 209.3 nm, which was slightly larger than the fetal stem cell-derived extracellular vesicles, and had an average concentration of 2.07×10⁸ particles/ml, which was about 12% of that of the fetal stem cell-derived extracellular vesicles (FIG. 4).
2) Analyzing extracellular vesicle marker expression using EXOVIEW®

Using EXOVIEW® (Nanoview Biosciences) equipment, which could quantitatively analyze the expression patterns of extracellular vesicle markers CD9, CD63, and CD81, the marker expression pattern of each of the extracellular vesicles was analyzed.

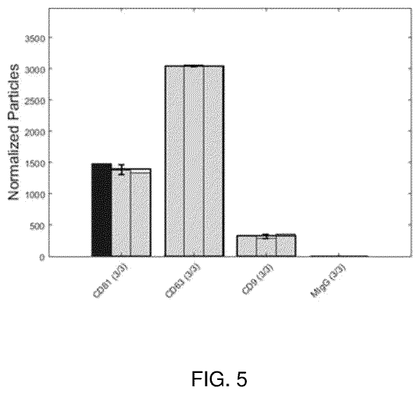

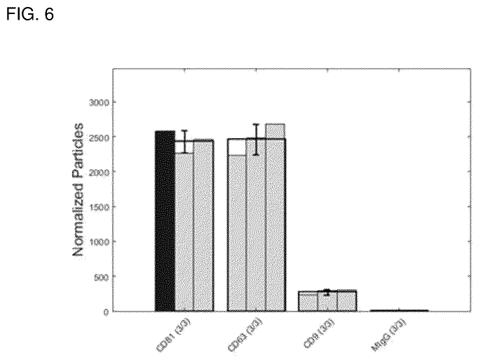

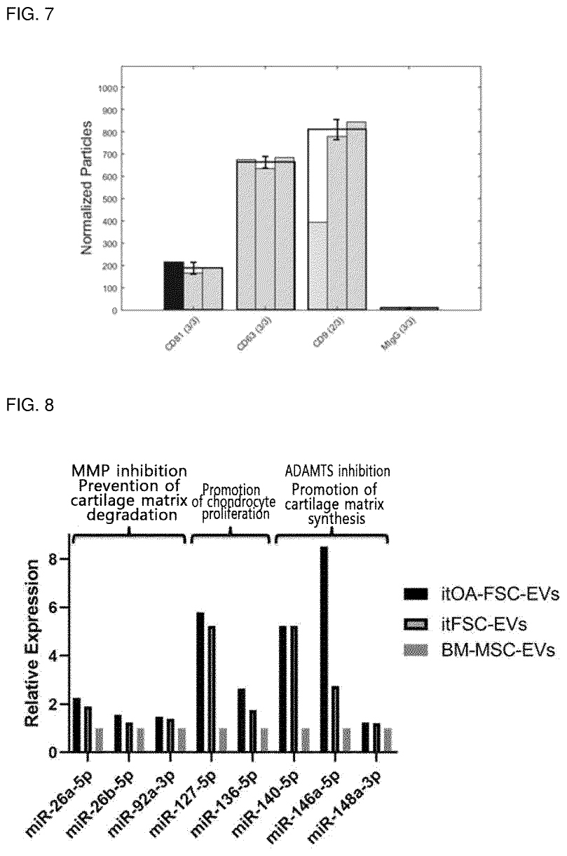

The fetal stem cell-derived extracellular vesicles, itOA-FSC-EVs and itFSC-EVs, showed a similar pattern in which CD9 marker expression was slightly lower than that of other extracellular vesicle markers, but CD63 expression was significantly increased in itOA-FSC-EVs unlike itFSC-EVs, which showed similar expression patterns of CD63 and CD81. This appeared to be due to the effect of the human chondrocytes and bone marrow-derived mesenchymal stem cells co-cultured extracellular vesicles (HChD/BM-MSC^{CO}-EVs) of Example 1 (FIGS. 5 and 6). Another control group, the bone marrow mesenchymal stem cell-derived extracellular vesicles (BM-MSC-EVs), showed a different marker expression pattern from the fetal stem cell extracellular vesicles, that is, CD9 and CD63 showed similar expression patterns, but CD81 showed a weak expression pattern (FIG. 7).
3) Comparison of cytokine and protein contents

The content of anti-inflammatory cytokines and cartilage regeneration-related proteins contained in each of the extracellular vesicles was analyzed using Human Cytokine Array (Raybiotech, QAH-CAA-1000).

Among the representative anti-inflammatory cytokines, IL-1ra, IL-4, IL-10, and IL-13 were below the detection limit in BM-MSC-EVs, and only TGF-31 was found to be contained at a low level.

In contrast, detectable anti-inflammatory cytokines were found to be contained at higher levels in itOA-FSC-EVs and itFSC-EVs, which are fetal stem cell-derived extracellular vesicles, than in BM-MSC-EVs. In particular, anti-inflammatory cytokines were found to be contained at similar or higher levels in itOA-FSC-EVs than in itFSC-EVs. In particular, TIMP-1 and TIMP-2, enzymes that inhibit MMP, an enzyme that degrades the extracellular matrix, were found to be contained at significantly higher levels in itOA-FSC-EVs than in BM-MSC-EVs (Table 2).

BMP4/5/7, FGF-2/7, and IGF-1, which are proteins that promote hyaline cartilage regeneration, were also found to be contained at higher levels in both itOA-FSC-EVs and itFSC-EVs than in BM-MSC-EVs. In particular, BMP4/5/7, FGF-2/7, and IGF-1 were found to be contained at similar or higher levels in itOA-FSC-EVs than in itFSC-EVs (Table 2).

TABLE 2

| | itOA-FSC-EVs | itFSC-EVs | BM-MSC-EVs |
|---|---|---|---|
| Cytokines | (pg/ml) | (pg/ml) | (pg/ml) |
| IL-1ra | 413.6 | 324.9 | 0.0 |
| IL-4 | 78.2 | 71.1 | 0.0 |
| IL-10 | 15.8 | 9.1 | 0.0 |
| IL-13 | 22.7 | 13.6 | 0.0 |
| TIMP-1 | 154,462.5 | 119,191.5 | 17,069.9 |
| TIMP-2 | 288,595.8 | 267,901.9 | 78,949.4 |
| TGF-β1 | 22,847.2 | 19,606.0 | 642.3 |
| BMP-4 | 2,759.4 | 1,019.3 | 80.1 |
| BMP-5 | 12,625.7 | 8,548.8 | 566.3 |
| BMP-7 | 299.9 | 202.6 | 61.5 |
| FGF-2 | 195.6 | 101.8 | 13.2 |
| FGF-7 | 526.2 | 440.6 | 116.1 |
| IGF-1 | 1,498.8 | 1,360.4 | 122.4 |

<Analysis of content of anti-inflammatory cytokines and proteins related to promotion of hyaline cartilage regeneration contained in itOA-FSC-EVs, itFSC-EVs, and BM-MSC-EVs>
4) Analyzing mRNA and miRNA expression related to cartilage regeneration and protection

The expression of miRNA and mRNA related to cartilage regeneration and protection, which were contained in each of the extracellular vesicles was analyzed by exosomal RNA analysis using next generation sequencing (NGS).

As a result, the expression levels of various miRNAs, such as miRNAs (miR-26a, miR-26b, and miR-92a) that protect cartilage by inhibiting the expression of genes that promote cartilage degradation, such as KPNA3, NOS2 and Wnt5A, miRNAs (miR-140-5p, miR-146a-5p and miR-148a-3p) that inhibit the degradation of cartilage matrix and promote its synthesis by inhibiting the expression of ADAMTS and MMP that are proteolytic enzymes that degrade the cartilage extracellular matrix, and miRNAs (miR-127-5p and miR-136) that promote the proliferation and migration of chondrocytes, which have been verified to play a role in cartilage regeneration and protection by being contained in the bone marrow stem cell-derived extracellular vesicles (BM-MSC-EVs) in various literatures, were all found to be higher in itOA-FSC-EVs and itFSC-EVs, which are fetal stem cell-derived extracellular vesicles, than in BM-MSC-EVs. In particular, it was verified that each miRNA was expressed at a similar or higher level in itOA-FSC-EVs than in itFSC-EVs (FIG. 8).

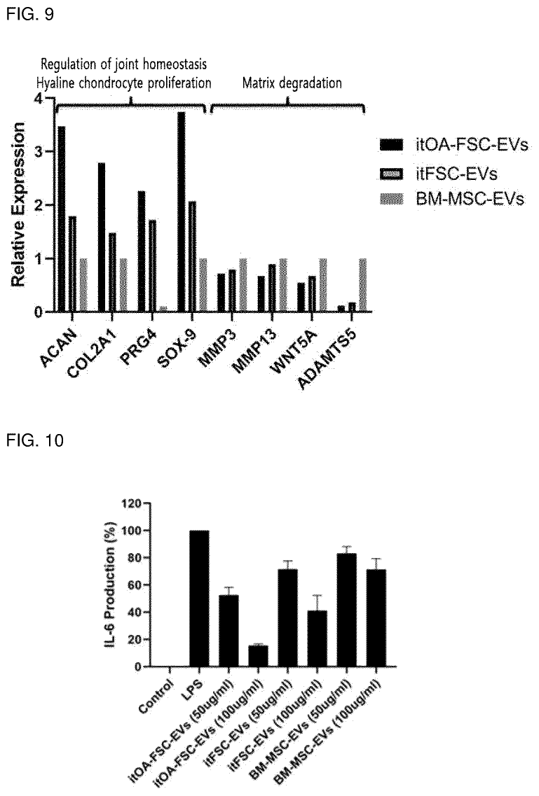

The expression levels of ACAN and COL2A1, which encode aggrecan and collagen II, the major proteins that make up the extracellular matrix of articular cartilage, and SOX-9 and PRG4, genes that maintain the homeostasis of the cartilage extracellular matrix, were also higher in itOA-FSC-EVs and itFSC-EVs, which are fetal stem cell-derived the extracellular vesicles, than in BM-MSC-EVs. In particular, it was verified that these were expressed at similar or higher levels in itOA-FSC-EVs than in itFSC-EVs (FIG. 9). The expression levels of MMP3, MMP13, WNT5A, and ADAMTS5, which are genes encoding proteins that degrade the extracellular matrix by activating MMPs that degrade the cartilage extracellular matrix, were verified to be all lower in both itOA-FSC-EVs and itFSC-EVs than in BM-MSC-EVs (FIG. 9).

### Example 5

Verifying Inflammation Relief Efficacy of Extracellular Vesicles for Osteoarthritis Treatment (itOA-MSC-EVs) Having Hyaline Cartilage Regeneration and Anti-Inflammatory Efficacies

In order to verify the inflammation relief efficacy of extracellular vesicles, an anti-inflammatory efficacy evaluation was conducted using human macrophages.

More specifically, human macrophages (PromoCell, Cat. #C-12914) stimulated with lipopolysaccharide (LPS) were treated with each of the extracellular vesicles, and the production pattern of inflammatory cytokines (IL-6, TNF-α, and PGE2) was analyzed and compared.

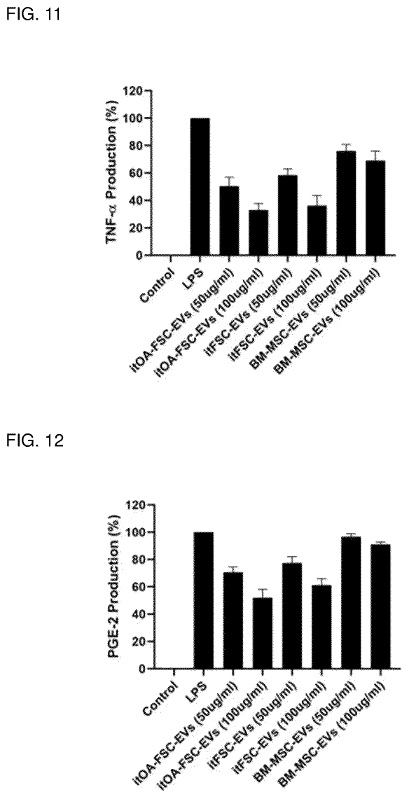

As a result, the production of IL-6, TNF-α, and PGE2 was significantly reduced in the treatment groups of itOA-FSC-EVs and itFSC-EVs, which are fetal stem cell-derived extracellular vesicles, than in the BM-MSC-EVs treatment group. In particular, it was verified that the production of inflammatory cytokines could be reduced to a similar or higher level in the itOA-FSC-EVs treatment group than in the itFSC-EVs treatment group (FIGS. 10 to 12).

### Example 6

Verifying Cartilage Extracellular Matrix Secretion Promoting Efficacy of Extracellular Vesicles for Osteoarthritis Treatment (itOA-MSC-EVs) Having Hyaline Cartilage Regeneration and Anti-Inflammatory Efficacies

In order to verify the cartilage extracellular matrix (ECM) secretion promoting efficacy of extracellular vesicles, the ECM secretion promoting efficacy was evaluated using human chondrocytes.

More specifically, human chondrocytes (PromoCell, Cat. #C-12710) were treated with each of the extracellular vesicles, and the contents of collagen II and aggrecan, which are the main components of the cartilage extracellular matrix, MMP13 and ADAMTP5, which are enzymes that degrade extracellular vesicles, and sex-determining region Y (SRY)-related protein 9 (SOX-9), which is a biomarker of hyaline cartilage, were analyzed and compared using RT-qPCR and Western blot.

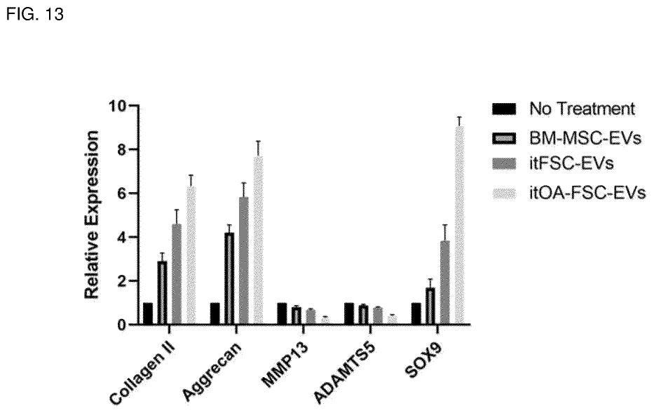

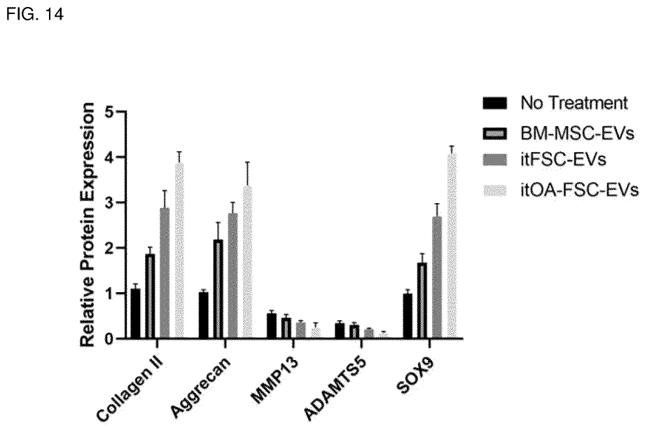

As a result, the expression of collagen II, aggrecan, and SOX9 was significantly increased in all the extracellular vesicle treatment groups compared to the control group (No Treatment). In particular, it was verified that the expression of collagen II, aggrecan, and SOX9 increased to the highest level in the itOA-FSC-EVs treatment group (FIGS. 13 and 14).

Compared to the control group (No Treatment), the expression of MMP13 and ADAMTS5 was significantly reduced in all the extracellular vesicle treatment groups. In particular, it was verified that the expression of MMP13 and ADAMTS5 decreased to the lowest level in the itOA-FSC-EVs treatment group (FIGS. 13 and 14).

### Example 7

Verifying In Vivo Hyaline Cartilage Regeneration Efficacy of Extracellular Vesicles for Osteoarthritis Treatment (itOA-FSC-EVs) Having Hyaline Cartilage Regeneration and Anti-Inflammatory Efficacies

In order to verify the osteoarthritis treating efficacy of extracellular vesicles, the hyaline cartilage regeneration efficacy was evaluated using an animal model of osteoarthritis induced by monosodium iodoacetate (MIA).

More specifically, 50 μl of MIA was injected into the right knee joint of anesthetized SD-rats, and osteoarthritis was found to be induced through visual observation of walking and knees for one week. Thereafter, 100 μl of itOA-FSC-EVs and itFSC-EVs were injected into the test groups intra-articularly.

After 4 and 12 weeks, the control, OA-induced (MIA-OA), and EV-treated (itOA-FSC-EVs and itFSC-EVs) groups were euthanized individually, then the knee joint was sampled, and the validity was evaluated by performing X-ray and micro-CT imaging and histological evaluation.

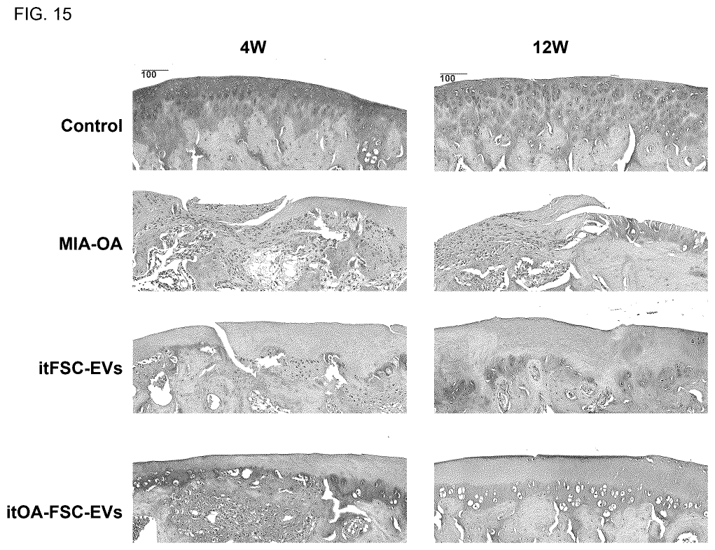

The knee joint sample tissue was stained with Safranin O (SFO), and histological evaluation was performed. As a result, it was found that the cartilage damage was so severe that the shape of normal cartilage tissue was barely recognizable in the OA-induced group (MIA-OA) compared to the control group, and it was found that cartilage regeneration was clearly progressing as the cartilage structure was maintained relatively intact in the EV treatment groups, itFSC-EVs and itOA-FSC-EVs, compared to the OA-induced group. In particular, it was found that cartilage regeneration progressed further in the itOA-FSC-EVs treatment group of the present invention compared to the itFSC-EVs treatment group, to a degree closer to that in the control group, and higher collagen II and proteoglycan contents were also observed in articular cartilage (FIG. 15).

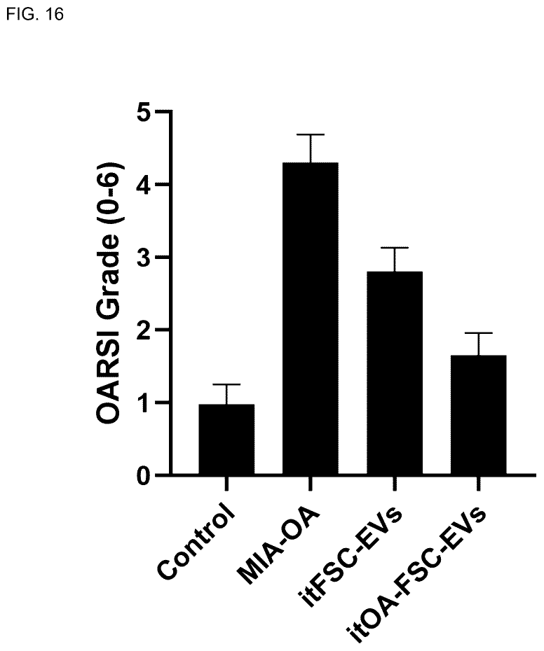

The degree of cartilage degeneration in the articular cartilage samples collected from each group was evaluated using the OARSI (osteoarthritis research society international) score, and as a result, the OA-induced group (MIA-OA) had a very high OARSI score of about 4.1 compared to the control group (Control) of about 0.9. It was found that the EV treatment groups, itFSC-EVs and itOA-FSC-EVs, had significantly reduced OARSI scores of about 2.8 and 1.7, respectively, compared to the OA-induced group (MIA-OA), and the OARSI score was lower in the itOA-FSC-EVs treatment group of the present invention than in the itFSC-EVs treatment group (FIG. 16).

Through the histological evaluation using immunohistochemical staining of articular cartilage samples collected from each group, the expression levels of collagen II, a marker of hyaline cartilage, aggrecan, a major cartilage extracellular matrix, MMP13, a cartilage-degrading enzyme, and collagen I, a marker of degenerative fibrocartilage, were compared.

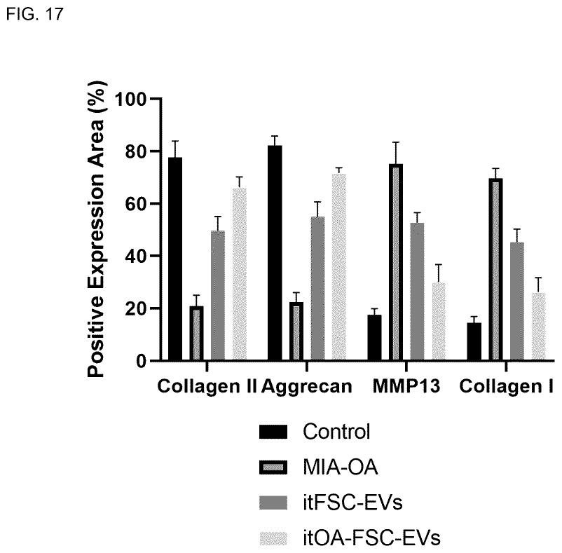

As a result, in the OA-induced group (MIA-OA), the expression of collagen II and aggrecan was significantly reduced, and the expression of MMP13 and collagen I was significantly increased compared to the control group. It was found that in the EV treatment groups, itFSC-EVs and itOA-FSC-EVs, the expression of collagen II and aggrecan was upregulated, and the expression of MMP13 and collagen I was reduced compared to the OA-induced group. In particular, it was found that the expression of collagen II and aggrecan was further upregulated, and the expression of MMP13 and collagen I was further reduced in the itOA-FSC-EVs treatment group of the present invention compared to the itFSC-EVs treatment group (FIG. 17).

Through these results, it has been verified that the immune-tolerized fetal stem cell-derived extracellular vesicles (itOA-FSC-EVs) having hyaline cartilage regeneration and anti-inflammatory efficacies have a higher level of efficacy in regenerating hyaline cartilage instead of degenerative fibrocartilage in osteoarthritic lesions compared to itFSC-EVs.

It has also been verified that itOA-FSC-EVs have superior therapeutic efficacies for osteoarthritis, including anti-inflammatory efficacy and cartilage protection efficacy through inhibition of MMP expression, compared to itFSC-EVs.

For reference, the present inventor determined that it would be greatly important to manufacture immune-tolerized extracellular vesicles containing human leukocyte antigen G (HLA-G) protein, which establishes an immune tolerance environment that perfectly protects the fetus from the mother's immune system during pregnancy in order for the extracellular vesicles, which are the main component of the composition for osteoarthritis treatment of the present invention, to be completely delivered to the damaged cartilage tissue without causing an immune response due to their own immunogenicity, and do not induce an immune response due to antigen mismatch.

To this end, the present inventor established ex vivo culture conditions that simulate the in vivo environment during pregnancy, which can induce the continuous secretion and expression of HLA-G protein, which establishes an immune tolerance environment that protects the fetus from the mother's immune system during pregnancy, through prior registered patents U.S. Pat. Nos. 11,566,220 B2, 11,771,720 B2, and 12,150,962 B2, which are incorporated into the present invention by reference in their entirety.

Through this, the present inventor induced immune tolerance properties that enable continuous expression and secretion of HLA-G protein in stem cells for producing extracellular vesicles for osteoarthritis treatment. The present inventor has newly established an ex vivo culture system for producing extracellular vesicles for osteoarthritis treatment having hyaline cartilage regeneration and anti-inflammatory efficacies by applying the ex vivo culture matrix that contains cartilage extracellular matrix components secreted through the co-culture of chondrocytes and bone marrow-derived mesenchymal stem cells to the established ex vivo culture conditions that simulate the in vivo environment during pregnancy in order to enhance the hyaline cartilage regeneration efficacy by extracellular vesicles.

It was verified that the immune-tolerized extracellular vesicles produced in the established ex vivo culture system do not induce immune rejection due to their own immunogenicity as well as have superior hyaline cartilage regeneration and inflammation relief efficacies compared to extracellular vesicles produced under general culture conditions and the established ex vivo culture conditions that simulate the in vivo environment during pregnancy, whereby the present invention has been completed.

The embodiments of the present invention described above have been described with reference to embodiments illustrated in the drawings to help understanding, but these are merely exemplary, and those skilled in the art will appreciate that various modifications and equivalent other embodiments are possible from this. Therefore, the true technical protection scope of the present invention should be defined by the appended claims.

1. A method for manufacturing immune-tolerized fetal stem cell-derived extracellular vesicles for osteoarthritis treatment, the method comprising:
a first step of obtaining extracellular vesicles containing cartilage extracellular matrix through the co-culture of human chondrocytes and bone marrow-derived mesenchymal stem cells;
a second step of obtaining the extracellular vesicles derived from immune-tolerized trophoblasts that continuously express and secrete HLA-G; and
a third step of inoculating fetal stem cells into an ex vivo culture matrix gel and subculturing the cells in a serum-free medium containing the human chondrocytes and bone marrow-derived mesenchymal stem cells co-cultured extracellular vesicles of the first step and the immune-tolerized trophoblast-derived extracellular vesicles of the second step under temperature change and vibration culture conditions similar to those in the body during pregnancy, wherein
the temperature change and vibration culture conditions similar to those in the body during pregnancy in the third step are a temperature change condition having a 5-day cycle in which the temperature changes to 36.5° C. at the 0 to 12th hour, 36.4° C. at the 12th to 36th hour, 36.3° C. at the 36th to 48th hour, 36.2° C. at the 48th to 60th hour, 36.0° C. at the 60th to 72nd hour, and to 37.0° C. at the 72nd to 120th hour and a vibration culture condition having a 24-hour cycle in which the vibration changes to 0 RPM at the 0 to 7th hour, 30 RPM at the 8th hour, 60 RPM at the 9th to 18th hour, 20 RPM at the 19th hour, and 0 RPM at the 20th to 24th hour.

2. The method for manufacturing immune-tolerized fetal stem cell-derived extracellular vesicles for osteoarthritis treatment according to claim 1, comprising:
a fourth step of inoculating the subcultured fetal stem cells into a culture plate, culturing the cells in a serum-free medium, and obtaining a culture supernatant; and
a fifth step of performing multi-stage filtration and separation on the culture supernatant.

3. The method for manufacturing immune-tolerized fetal stem cell-derived extracellular vesicles for osteoarthritis treatment according to claim 1, wherein the extracellular vesicles obtained through the co-culture of human chondrocytes and bone marrow-derived mesenchymal stem cells of the first step are manufactured through
step 1-1 of inoculating human chondrocytes and bone marrow-derived mesenchymal stem cells onto the upper and lower parts of a co-culture plate, respectively;
step 1-2 of performing the co-culture in a serum-free medium and obtaining a culture supernatant; and
step 1-3 of performing multi-stage filtration and separation on the culture supernatant.

4. The method for manufacturing immune-tolerized fetal stem cell-derived extracellular vesicles for osteoarthritis treatment according to claim 1, wherein the extracellular vesicles obtained through the co-culture of human chondrocytes and bone marrow-derived mesenchymal stem cells of the first step contain collagen types II, VI, IX, and XII, fibronectin, proteoglycan link protein 1 (HPLN-1), extracellular matrix protein-1 (ECM-1), and tissue metalloproteinase inhibitor 1 (TIMP1), which protect the cartilage extracellular matrix and promote secretion of the cartilage extracellular matrix in fetal stem cells to maintain cartilage homeostasis.

5. The method for manufacturing immune-tolerized fetal stem cell-derived extracellular vesicles for osteoarthritis treatment according to claim 1, wherein the extracellular vesicles derived from immune-tolerized trophoblasts that continuously express and secrete HLA-G of the second step are manufactured through
step 2-1 of obtaining extracellular vesicles through the co-culture of human amniotic membrane-derived mesenchymal stem cells and amniotic fluid-derived mesenchymal stem cells;
step 2-2 of inoculating human trophoblasts into an ex vivo culture matrix gel containing hyaluronic acid and the extracellular vesicles of step 2-1; and
step 2-3 of culturing the trophoblasts under temperature change and vibration culture conditions similar to those in the body during pregnancy, wherein
the temperature change and vibration culture conditions similar to those in the body during pregnancy in step 2-3 are a temperature change condition having a 5-day cycle in which the temperature changes to 36.5° C. at the 0 to 12th hour, 36.4° C. at the 12th to 36th hour, 36.3° C. at the 36th to 48th hour, 36.2° C. at the 48th to 60th hour, 36.0° C. at the 60th to 72nd hour, and to 37.0° C. at the 72nd to 120th hour and a vibration culture condition having a 24-hour cycle in which the vibration changes to 0 RPM at the 0 to 7th hour, 30 RPM at the 8th hour, 60 RPM at the 9th to 18th hour, 20 RPM at the 19th hour, and 0 RPM at the 20th to 24th hour.

6. The method for manufacturing immune-tolerized fetal stem cell-derived extracellular vesicles for osteoarthritis treatment according to claim 1, wherein the ex vivo culture matrix gel of the third step contains:
extracellular vesicles obtained through the co-culture of human amniotic membrane-derived mesenchymal stem cells and amniotic fluid-derived mesenchymal stem cells; and
hyaluronic acid.

7. The method for manufacturing immune-tolerized fetal stem cell-derived extracellular vesicles for osteoarthritis treatment according to claim 1, wherein the ex vivo culture matrix gel of the third step maintains an acidic condition of pH 6 to 7.

8. The method for manufacturing immune-tolerized fetal stem cell-derived extracellular vesicles for osteoarthritis treatment according to claim 2, wherein the subcultured fetal stem cells of the fourth step have HLA-G protein present on a cell surface or in a culture supernatant.

9. The method for manufacturing immune-tolerized fetal stem cell-derived extracellular vesicles for osteoarthritis treatment according to claim 1, wherein the obtained fetal stem cell-derived extracellular vesicles promote differentiation into hyaline cartilage and reduce joint inflammation.

10. A pharmaceutical composition for osteoarthritis treatment comprising immune-tolerized fetal stem cell-derived extracellular vesicles that are manufactured by the method according to claim 1, wherein
the extracellular vesicles contain fibroblast growth factor-2 (FGF-2), fibroblast growth factor-7 (FGF-7), insulin-like growth factor-1 (IGF-1), transforming growth factor-beta 1 (TGF-β1), bone morphogenetic protein-4 (BMP-4), bone morphogenetic protein-5 (BMP-5), and bone morphogenetic protein-7 (BMP-7), and include HLA-G protein.

11. The pharmaceutical composition for osteoarthritis treatment comprising immune-tolerized fetal stem cell-derived extracellular vesicles according to claim 10, wherein the extracellular vesicles additionally contain interleukin-1 receptor antagonist (IL-1ra), interleukin-4 (IL-4), interleukin-10 (IL-10), and interleukin-13 (IL-13).

12. The pharmaceutical composition for osteoarthritis treatment comprising immune-tolerized fetal stem cell-derived extracellular vesicles according to claim 10, wherein the extracellular vesicles additionally contain tissue metalloproteinase inhibitor 1 (TIMP1) and tissue metalloproteinase inhibitor 2 (TIMP2).

13. The pharmaceutical composition for osteoarthritis treatment comprising immune-tolerized fetal stem cell-derived extracellular vesicles according to claim 10, wherein the extracellular vesicles additionally contain miR-26a, miR-26b, miR-92a, miR-127, miR-136, miR-140, miR-146a, and miR-148.

14. The pharmaceutical composition for osteoarthritis treatment comprising immune-tolerized fetal stem cell-derived extracellular vesicles according to claim 10, wherein the extracellular vesicles additionally contain aggrecan, collagen II, proteoglycan 4 (PRG4), and sex-determining region Y (SRY)-related protein 9 (SOX9).

15. The pharmaceutical composition for osteoarthritis treatment comprising immune-tolerized fetal stem cell-derived extracellular vesicles according to claim 10, wherein the extracellular vesicles are established by culturing fetal stem cells isolated from amniotic fluid collected for amniocentesis in early pregnancy in an ex vivo culture matrix gel that induces immune tolerance properties.
